(19) United States
(12) Patent Application Publication
(10) Pub. No.: US 2025/0127181 A1
(43) Pub. Date: Apr. 24, 2025

(54) NON-ACIDIFIED FERMENTED DAIRY PRODUCT AND PREPARATION METHOD THEREOF

(71) Applicant: University Of Shanghai For Science And Technology, Shanghai (CN)

(72) Inventors: Lianzhong AI, Shanghai (CN); Yijin YANG, Shanghai (CN); Yongjun XIA, Shanghai (CN); Guangqiang WANG, Shanghai (CN); Xin SONG, Shanghai (CN); Zhiqiang XIONG, Shanghai (CN); Hui ZHANG, Shanghai (CN)

(21) Appl. No.: 18/901,403

(22) Filed: Sep. 30, 2024

(30) Foreign Application Priority Data
Oct. 24, 2023 (CN) 202311380640.5

(51) Int. Cl. A23C 9/12; A23C 3/02; C12N 1/18; C12R 1/865

(52) U.S. Cl. CPC A23C 9/1203; A23B 11/12; C12N 1/185; C12R 2001/865

(57) ABSTRACT

A non-acidified fermented dairy product and a preparation method thereof are provided, relating to the technical field of fermented dairy products processing. The preparation method of the non-acidified fermented dairy product includes: inoculating a fermentation inoculum into cow milk, and performing a two-stage fermentation to obtain the non-acidified fermented milk; wherein the two-stage fermentation comprises a first-stage fermentation and a second-stage fermentation; a rotation speed of the first-stage fermentation is 180-250 revolutions per minute, and a duration is 10-20 hours; a rotation speed of the second-stage fermentation is 100-160 revolutions per minute, and a duration is 8-16 hours.

### CROSS-REFERENCE TO RELATED APPLICATIONS

This application claims priority to Chinese Patent Application No. 202311380640.5, filed on Oct. 24, 2023, the contents of which are hereby incorporated by reference.

### TECHNICAL FIELD

The present disclosure relates to the technical field of fermented dairy products processing, and in particular to a non-acidified fermented dairy product and a preparation method thereof.

### BACKGROUND

Milk is recognized worldwide as a very nutritious food for containing rich proteins, fats, carbohydrates and minerals, especially cows' milk, which is a drink containing casein, whey protein, milk fat, as well as trace elements and many other essential nutrients for human health.

Based on the original nutritional value of milk, the fermented dairy products obtained by adding strains to milk as substrate are beneficial to intestinal peristalsis and improving immunity. Some protein is decomposed into peptides and amino acids, and some fat and lactose are also decomposed into smaller compounds, thus enabling the fermented dairy products to be more easily absorbed by human body.

Recent years have witnessed a gradual increase in people's health awareness with the improvement of their living standards, and the demand for nutritional and healthcare components of food has become increasingly strong. The existing milk beverages on the market are currently available in many types, mainly fresh milk, fermented milk, fruit milk and other types, which are welcomed by consumers. Among them, fermented milk products have become one of the daily consumption of the public, which has both nutritional value and health care functions. The vast majority of fermented dairy products on the market so far are prepared by using lactic acid bacteria for acidification and fermentation. In the fermentation process, lactic acid and diacetyl are produced by lactic acid bacteria, which give the fermented dairy products a unique flavor and texture; however, some consumers are not comfortable with the flavor of yogurt, and it is necessary to develop non-acidified fermented dairy products to diversify the dairy products market.

### SUMMARY

The present disclosure aims to provide a non-acidified fermented dairy product and a preparation method thereof, so as to solve the problems existing in the prior art. By adopting the method of the present disclosure, a non-acidified fermented dairy product with good flavor and taste is obtained, thus diversifying the dairy product market.

In order to achieve the above objectives, the present disclosure provides the following schemes.

The present disclosure provides a preparation method of a non-acidified fermented milk, including following steps:
inoculating a fermentation inoculum into cow milk, and performing a two-stage fermentation to obtain the non-acidified fermented milk;
the two-stage fermentation includes a first-stage fermentation and a second-stage fermentation; a rotation speed of the first-stage fermentation is 180-250 revolutions per minute (rpm), and a duration is 10-20 hours (h); a rotation speed of the second-stage fermentation is 100-160 rpm, and a duration is 8-16 h.

Optionally, an active ingredient of the fermentation inoculum is Saccharomyces cerevisiae BR14; and an inoculation amount of the fermentation inoculum is 1%-10% of a volume of the cow milk.

Optionally, an OD₆₀₀ value of the fermentation inoculum is 1.

Optionally, a preparation method of the fermentation inoculum includes following steps:
activating the Saccharomyces cerevisiae BR14, and then carrying out seed liquid culture and fermentation culture.

Optionally, a medium used for activating is a Yeast Extract-Peptone-Dextrose (YPD) solid medium, with a duration of 48 h and a temperature of 28 degrees Celsius (° C.);
a medium used for seed liquid culture is a YPD liquid culture medium, with a duration of 24 h and a temperature of 28° C.; and
the fermentation culture medium adopts the YPD liquid culture medium, with a duration of 12 h, a temperature of 28° C., a rotation speed of 150 rpm, and an inoculation amount of 5% of a volume of the YPD liquid culture medium.

Optionally, the cow milk is cow milk obtained after sterilization.

Optionally, a temperature of the sterilization is 143-158° C. and a duration is 0.05-0.5 second(s).

The present disclosure provides a non-acidified fermented milk, and the non-acidified fermented milk is obtained by two-stage fermentation of cow milk with Saccharomyces cerevisiae BR14 as a fermentation inoculum.

The present disclosure also provides an application of the non-acidified fermented milk in preparing non-acidified fermented milk products, including dairy products and freeze-dried powder.

Optionally, when the products are dairy products, a step of pasteurizing the non-acidified fermented milk is further included; and

when the products are freeze-dried powder, a step of freeze-drying the non-acidified fermented milk is further included.

The present disclosure achieves the following technical effects.

The present disclosure provides a preparation method of a non-acidified fermented milk, which includes the following steps: inoculating a fermentation inoculum into cow milk, and performing a two-stage fermentation to obtain the non-acidified fermented milk; the two-stage fermentation includes a first-stage fermentation and a second-stage fermentation; the rotation speed of the first-stage fermentation is 180-250 rpm, and the duration is 10-20 h; the rotation speed of the second-stage fermentation is 100-160 rpm, and the duration is 8-16 h. According to the present disclosure, the non-acidified fermented milk is obtained by using a fermentation strain as the fermentation inoculum (especially Saccharomyces cerevisiae BR14) and raw cow milk as the raw material; the obtained product retains the original liquid flowing state of the cow milk and has good flavor and texture, with significantly reduced lactose content in cow milk and improved digestibility of milk proteins. Also, the present disclosure ensures that the fermentation strain (Saccharomyces cerevisiae BR14) proliferates effectively in cow milk by using stepwise aerobic fermentation, and the lactose content in cow milk is reduced by more than 30%; the rotation speed of the first stage of fermentation is relatively high, which effectively promotes the yeast to proliferate and to consume lactose in the cow milk; the rotation speed of the second stage of fermentation is lower, thus effectively promoting the synthesis of flavor substances; and owing to the aerobic fermentation, the content of ethanol in the final fermented milk is extremely low, with a content of only 0.002 milligram per milliliter (mg/mL).

Moreover, the present disclosure takes Saccharomyces cerevisiae BR14 as a fermentation inoculum, since Saccharomyces cerevisiae BR14 secretes proteases that moderately degrade milk proteins during growth, yet the total nitrogen content of the cow milk is not affected, nor does it affect the suspension of protein micelles and change the original liquid nature of cow milk; Saccharomyces cerevisiae BR14 may slightly degrade milk fat and produce a variety of volatiles, producing good flavor properties; in addition, Saccharomyces cerevisiae BR14 is isolated from the lees of yellow wine and has excellent probiotic properties and good anti-inflammatory activity, which enable the fermented milk to have better functional properties.

Besides, the content of casein in the non-acidified fermented milk prepared by the preparation method of the present disclosure is less, which may reduce the cow milk allergy symptoms of consumers.

### Description Information of Biological Deposit:

Saccharomyces cerevisiae BR14, deposited in the China General Microbiological Culture Collection Center (CGMCC), with a deposit address of Institute of Microbiology, China Academy of Sciences, No. 3, No. 1 Courtyard of Beichen West Road, Chaoyang District, Beijing, a postcode of 100101, a deposit date of Jul. 13, 2020 and a deposit number of CGMCC No. 20352.

### BRIEF DESCRIPTION OF THE DRAWINGS

In order to explain the embodiments of the present disclosure or the technical scheme in the prior art more clearly, the drawings needed in the embodiments are briefly introduced below. Obviously, the drawings described below are only some embodiments of the present disclosure, and other drawings may be obtained according to these drawings without creative work for ordinary people in the field.

FIG. 1A shows the contents of aldehydes, alkenes, alcohols, alkanes and acids in unfermented cow milk.

FIG. 1B shows the contents of aldehydes, esters, ketones, alcohols, alkanes and acids in pasteurized non-acidified fermented dairy products.

FIG. 1C shows a comparison of flavor substance composition in unfermented cow milk and pasteurized non-acidified fermented dairy products.

FIG. 2 is a sodium dodecyl sulfate polyacrylamide gel electrophoresis (SDS-PAGE) diagram of pasteurized non-acidified fermented dairy products, in which lane 1 is unfermented cow milk and lane 2 is the pasteurized non-acidified fermented dairy products.

FIG. 3 shows the polypeptide analysis of pasteurized non-acidified fermented dairy products, in which FM is the pasteurized non-acidified fermented dairy product, Raw Milk is unfermented cow milk and the retention time is retention time.

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges may be independently included or excluded from the range.

Unless otherwise specified, all technical and scientific terms used herein have the same meaning as commonly understood by one of ordinary skill in the art to which this present disclosure relates. Although the present disclosure only describes the preferred methods and materials, any methods and materials similar or equivalent to those described herein may also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present disclosure without departing from the scope or spirit of the present disclosure. Other embodiments will be apparent to the skilled person from the description of the present disclosure. The description and embodiments of that present disclosure are exemplary only.

The terms “including”, “comprising”, “having” and “containing” used in this specification are all open terms, which means including but not limited to.

The Saccharomyces cerevisiae BR14 used in the present disclosure is separated from lees of yellow rice wine, and has excellent probiotic characteristics. Studies have shown that the Saccharomyces cerevisiae BR14 has good fermentation characteristics and good acid and bile salt tolerance. The Saccharomyces cerevisiae BR14 has been deposited in the China General Microbiological Culture Collection Center on Aug. 20, 2020, with the deposit number of CGMCC No. 20352.

### Embodiment 1 Preparation of Pasteurized Non-Acidified Fermented Dairy Product

The preparation method of the non-acidified fermented dairy product provided by this embodiment is as follows:
step 1: a certain amount of raw cow milk is directly sterilized by steam immersion (with a sterilization temperature of 143-158° C. and a sterilization duration of 0.05-0.5 s) and quickly cooled to 28° C. after the sterilization; and
step 2: the Saccharomyces cerevisiae BR14 is activated, specifically including the following steps: inoculating Saccharomyces cerevisiae BR14 into the YPD solid culture medium, and culturing at 28° C. for 48 h to obtain activated Saccharomyces cerevisiae BR14, where the YPD solid medium includes 20 grams per liter (g/L) glucose, 20 g/L plant soybean peptone, 10 g/L yeast extract powder and 1.5 wt. % bacterial agar powder, and is sterilized at 115° C. for 20 min;
the activated Saccharomyces cerevisiae BR14 is inoculated into 10 mL of liquid culture medium YPD, and cultured at 28° C. for 24 h to obtain the seed liquid of the Saccharomyces cerevisiae BR14; among them, the components of YPD liquid culture medium are as follows: 20 g/L glucose, 20 g/L plant soybean peptone and 10 g/L yeast, and sterilized at 115° C. for 20 min;
the obtained seed liquid is inoculated into 100 mL YPD liquid culture medium with an inoculation amount of 5% of the volume of YPD liquid culture medium, and cultured at 28° C. and 150 rpm for 12 h; after the culture, the thalli are collected and the OD₆₀₀ value of Saccharomyces cerevisiae BR14 is adjusted to 1 with sterile water;
step 3: inoculation: the Saccharomyces cerevisiae BR14 in step 2 is inoculated into the sterilized cow milk and mixed, where the inoculation amount is 1% of the volume of the sterilized cow milk;
step 4: two-stage fermentation: two-stage fermentation is carried out after uniform stirring, specifically: the first-stage fermentation: the rotation speed of the fermentation is controlled at 180 rpm, and fermentation is carried out for 10 h; the second-stage fermentation: the rotation speed of the fermentation is controlled at 100 rpm, and the fermentation is carried out for 8 h to obtain the non-acidified fermented milk; the lactose content of non-acidified fermented milk is 51.3 mg/L, which is 13.5% lower than that of unfermented cow milk, and the pH value is 6.65; the fermented milk has a strong fragrance and a slight floral fragrance; and
step 5: the non-acidified fermented milk after fermentation is homogenized, and then pasteurized at 80° C. for 15 s to obtain the pasteurized non-acidified fermented dairy product.

### Embodiment 2 Preparation of Pasteurized Non-Acidified Fermented Dairy Product

The preparation method of the non-acidified fermented dairy products provided by this embodiment is as follows:
step 1: a certain amount of raw cow milk is directly sterilized by steam immersion (sterilization temperature is 143-158° C., sterilization duration is 0.05-0.5 s), and quickly cooled to 28° C. after sterilization;
step 2: the Saccharomyces cerevisiae BR14 is activated, specifically including the following steps: inoculating Saccharomyces cerevisiae BR14 into the YPD solid culture medium, and culturing at 28° C. for 48 h to obtain activated Saccharomyces cerevisiae BR14, where the YPD solid medium includes 20 g/L glucose, 20 g/L plant soybean peptone, 10 g/L yeast extract powder and 1.5 wt. % bacterial agar powder, with sterilization at 115° C. for 20 min;
the activated Saccharomyces cerevisiae BR14 is inoculated into 10 mL of liquid culture medium YPD, and cultured at 28° C. for 24 h to obtain the seed liquid of the Saccharomyces cerevisiae BR14; among them, the components of YPD liquid culture medium are as follows: 20 g/L glucose, 20 g/L plant soybean peptone and 10 g/L yeast, and sterilized at 115° C. for 20 min;
the obtained seed liquid is inoculated into 100 mL YPD liquid culture medium with an inoculation amount of 5% of the volume of YPD liquid culture medium, and cultured at 28° C. and 150 rpm for 12 h; after the culture, the thalli are collected and the OD₆₀₀ value of Saccharomyces cerevisiae BR14 is adjusted to 1 with sterile water;
step 3: inoculation: the Saccharomyces cerevisiae BR14 in step 2 is inoculated into the sterilized cow milk and mixed, where the inoculation amount is 5% of the volume of the sterilized cow milk;
step 4: two-stage fermentation: the two-stage fermentation is carried out after uniform stirring, specifically: the first-stage fermentation: the rotation speed of the fermentation is controlled at 210 rpm, and fermentation is carried out for 16 h; the second-stage fermentation: the rotation speed of the fermentation is controlled at 140 rpm, and the fermentation is carried out for 12 h to obtain the non-acidified fermented milk; the lactose content of non-acidified fermented milk is 40.3 mg/L, which is 32.07% lower than that of unfermented cow milk, and the pH value is 6.68; the fermented milk has a strong fragrance and a slight floral fragrance; and
step 5: the non-acidified fermented milk product after fermentation is homogenized, and then pasteurized at 80° C. for 15 s to obtain the pasteurized non-acidified fermented dairy product.

### Embodiment 3 Preparation of Pasteurized Non-Acidified Fermented Dairy Product

The preparation method of the non-acidified fermented dairy products provided by this embodiment is as follows:
step 1: a certain amount of raw cow milk is directly sterilized by steam immersion (sterilization temperature is 143-158° C., sterilization duration is 0.05-0.5 s), and quickly cooled to 28° C. after sterilization;
step 2: the Saccharomyces cerevisiae BR14 is activated, specifically including the following steps: inoculating Saccharomyces cerevisiae BR14 into the YPD solid culture medium, and culturing at 28° C. for 48 h to obtain activated Saccharomyces cerevisiae BR14, where the YPD solid medium includes 20 g/L glucose, 20 g/L plant soybean peptone, 10 g/L yeast extract powder and 1.5 wt. % bacterial agar powder, with sterilization at 115° C. for 20 min;
the activated Saccharomyces cerevisiae BR14 is inoculated into 10 mL of liquid culture medium YPD, and cultured at 28° C. for 24 h to obtain the seed liquid of the Saccharomyces cerevisiae BR14; among them, the components of the adopted YPD liquid culture medium are as follows: 20 g/L glucose, 20 g/L plant soybean peptone and 10 g/L yeast, and sterilized at 115° C. for 20 min;
the obtained seed liquid is inoculated into 100 mL YPD liquid culture medium with an inoculation amount of 5% of the volume of YPD liquid culture medium, and cultured at 28° C. and 150 rpm for 12 h; after the culture, the thalli are collected and the OD₆₀₀ value of Saccharomyces cerevisiae BR14 is adjusted to 1 with sterile water;
step 3: inoculation: the Saccharomyces cerevisiae BR14 in step 2 is inoculated into the sterilized cow milk and mixed, where the inoculation amount is 10% of the volume of the sterilized cow milk;
step 4: two-stage fermentation: two-stage fermentation is carried out after uniform stirring, specifically: the first-stage fermentation: the rotation speed of the fermentation is controlled at 250 rpm, and fermentation is carried out for 20 h; the second-stage fermentation: the rotation speed of the fermentation is controlled at 160 rpm, and the fermentation is carried out for 16 h to obtain the non-acidified fermented milk; the lactose content of non-acidified fermented milk is 40.5 mg/L, which is 31.73% lower than that of unfermented cow milk, and the pH value is 6.85; the fermented milk has a strong fragrance and a slight floral fragrance; and
step 5: the non-acidified fermented milk product after fermentation is homogenized, and then pasteurized at 80° C. for 15 s to obtain the pasteurized non-acidified fermented dairy product.

### Embodiment 4 Preparation of Non-Acidified Fermented Freeze-Dried Product

The preparation method of non-acidified fermented dairy products provided by this embodiment is as follows:
step 1: a certain amount of raw cow milk is directly sterilized by steam immersion (sterilization temperature is 143-158° C., sterilization duration is 0.05-0.5 s), and quickly cooled to 28° C. after sterilization;
step 2: the Saccharomyces cerevisiae BR14 is activated, specifically including the following steps: inoculating Saccharomyces cerevisiae BR14 into the YPD solid culture medium, and culturing at 28° C. for 48 h to obtain activated Saccharomyces cerevisiae BR14, where the YPD solid medium includes 20 g/L glucose, 20 g/L plant soybean peptone, 10 g/L yeast extract powder and 1.5 wt. % bacterial agar powder, with sterilization at 115° C. for 20 min;
the activated Saccharomyces cerevisiae BR14 is inoculated into 10 mL of liquid culture medium YPD, and cultured at 28° C. for 24 h to obtain the seed liquid of the Saccharomyces cerevisiae BR14; among them, the components of the adopted YPD liquid culture medium are as follows: 20 g/L glucose, 20 g/L plant soybean peptone and 10 g/L yeast, and sterilized at 115° C. for 20 min;
the obtained seed liquid is inoculated into 100 mL YPD liquid culture medium with an inoculation amount of 5% of the volume of YPD liquid culture medium, and cultured at 28° C. and 150 rpm for 12 h; after the culture, the thalli are collected and the OD₆₀₀ value of Saccharomyces cerevisiae BR14 is adjusted to 1 with sterile water;
step 3: inoculation: the Saccharomyces cerevisiae BR14 in step 2 is inoculated into the sterilized cow milk and mixed, where the inoculation amount is 10% of the volume of the sterilized cow milk;
step 4: two-stage fermentation: two-stage fermentation is carried out after uniform stirring; the first-stage fermentation: the rotation speed of the fermentation is controlled at 210 rpm, and fermentation is carried out for 16 h; the second-stage fermentation: the rotation speed of the fermentation is controlled at 140 rpm, and the fermentation is carried out for 12 h to obtain the non-acidified fermented milk, which has a strong fragrance and a slight floral fragrance; and
step 5: the non-acidified fermented dairy products after fermentation is freeze-dried to obtain non-acidified fermented milk freeze-dried powder, which is used for the development of portable brewing products.

### Embodiment 5 Flavor Analysis of Pasteurized Non-Acidified Fermented Dairy Product

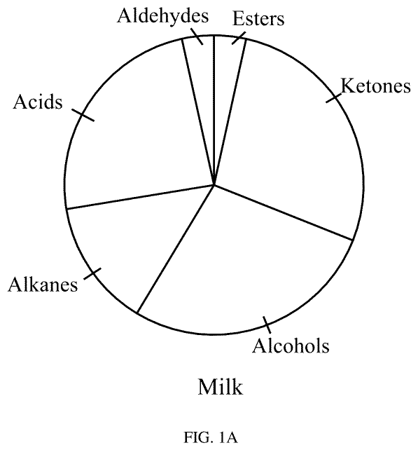

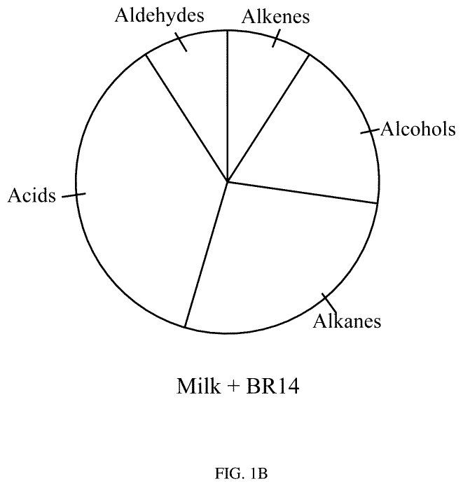

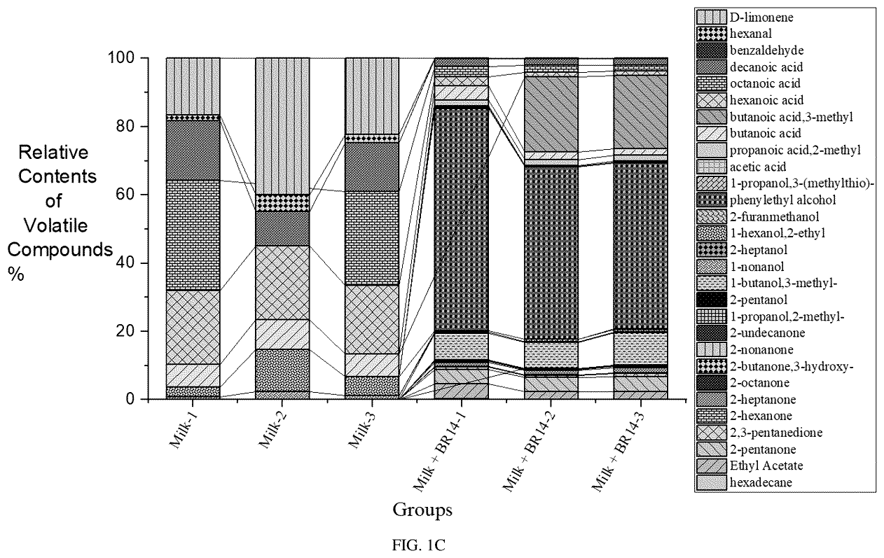

In this embodiment, the pasteurized non-acidified fermented dairy product prepared by the method of Embodiment 3 is used, and the unfermented milk and pasteurized non-acidified fermented dairy product are analyzed by Gas Chromatography-Mass Spectrometry (GC-MS); as may be seen from the results as shown in FIG. 1A-FIG. 1C, the Saccharomyces cerevisiae BR14 has a significant effect on the flavor of the cow milk. The FIG. 1A and FIG. 1B show that the types and contents of alcohols, acids and ketones in pasteurized non-acidified fermented dairy products are increased significantly, while the contents of aldehydes is decreased significantly, and further analysis (FIG. 1C) proves that the flavor composition of pasteurized non-acidified fermented dairy product is significantly different from that of unfermented cow milk. The main aroma components in pasteurized non-acidified fermented dairy product are phenylethanol, 3-methyl-butyric acid and 3-methyl-butanol, among which phenylethanol has the highest content, which gives the fermented milk a rose fragrance.

### Embodiment 6 Protein Degradation Analysis of Pasteurized Non-Acidified Fermented Dairy Product

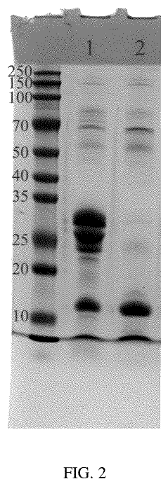

In this embodiment, the pasteurized non-acidified fermented dairy products are prepared by the method of Embodiment 3, and the proteins and peptides of the non-acidified fermented milk are analyzed by Sodium dodecyl-sulfate polyacrylamide gel electrophoresis (SDS-PAGE) and High Performance Liquid Chromatography (HPLC). The results of SDS-PAGE (FIG. 2) show that the content of casein (molecular weight 25-30 kDa) in cow milk is decreased after BR14 fermentation. Casein is one of the substances that often cause allergies in cow milk, and the reduction of its content is beneficial to reduce the allergic symptoms of cow milk.

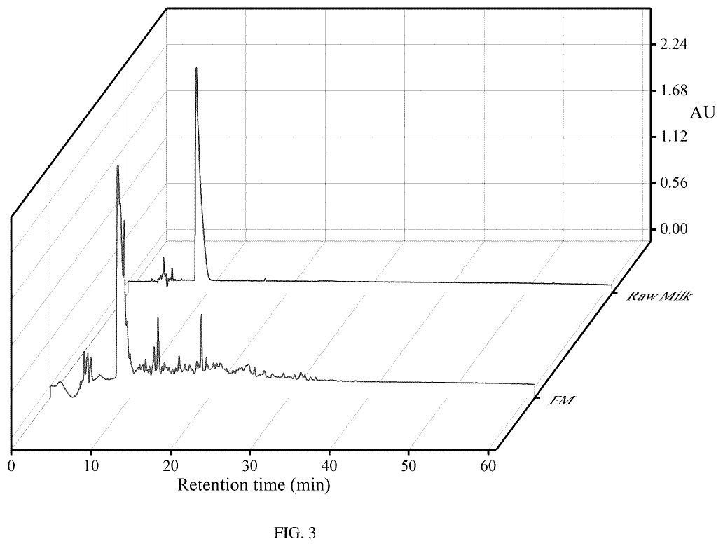

Furthermore, the peptides in pasteurized non-acidified fermented dairy product are analyzed by HPLC. According to the results as shown in FIG. 3, the types and contents of peptides in pasteurized non-acidified fermented dairy product are increased significantly compared with the unfermented cow milk. Polypeptide has good absorbability and functionality, which gives the fermented milk better physiological activity.

### Comparative Embodiment 1 Effect of Single-Stage Fermentation on Pasteurized Non-Acidified Fermented Dairy Product

The preparation method of non-acidified fermented dairy products provided by this comparative embodiment is as follows:
step 1: a certain amount of raw cow milk is directly sterilized by steam immersion (sterilization temperature is 143-158° C., sterilization duration is 0.05-0.5 s), and quickly cooled to 28° C. after sterilization;
step 2: the Saccharomyces cerevisiae BR14 is activated, specifically including the following steps: inoculating Saccharomyces cerevisiae BR14 into the YPD solid culture medium, and culturing at 28° C. for 48 h to obtain activated Saccharomyces cerevisiae BR14, where the YPD solid medium includes 20 g/L glucose, 20 g/L plant soybean peptone, 10 g/L yeast extract powder and 1.5 wt. % bacterial agar powder, with sterilization at 115° C. for 20 min;
the activated Saccharomyces cerevisiae BR14 is inoculated into 10 mL of liquid culture medium YPD, and cultured at 28° C. for 24 h to obtain the seed liquid of the Saccharomyces cerevisiae BR14; among them, the components of the adopted YPD liquid culture medium are as follows: 20 g/L glucose, 20 g/L plant soybean peptone and 10 g/L yeast, and sterilized at 115° C. for 20 min;
the obtained seed liquid is inoculated into 100 mL YPD liquid culture medium with an inoculation amount of 5% of the volume of YPD liquid culture medium, and cultured at 28° C. and 150 rpm for 12 h; after the culture, the thalli are collected and the OD₆₀₀ value of Saccharomyces cerevisiae BR14 is adjusted to 1 with sterile water;
step 3: inoculation: the Saccharomyces cerevisiae BR14 in step 2 is inoculated into the sterilized cow milk and mixed, where the inoculation amount is 5% of the volume of the sterilized cow milk;
step 4: single-stage fermentation: the fermentation is carried out after evenly stirring, specifically: the rotation speed of the fermentation is controlled at 180 rpm, and the fermentation is carried out for 12 h to obtain the non-acidified fermented milk; the lactose content in the non-acidified fermented milk is 52.8 mg/L, which is 10.97% lower than that of unfermented cow milk, and the pH value is 6.88, with a light milk flavor and a slight lipolytic smell; and
step 5: the non-acidified fermented milk product after fermentation is homogenized, and then pasteurized at 80° C. for 15 s to obtain the pasteurized non-acidified fermented dairy product.

### Comparative Embodiment 2 Effect of Single-Stage Fermentation on Pasteurized Non-Acidified Fermented Dairy Product

The preparation method of the non-acidified fermented dairy product provided by this comparative embodiment is as follows:
step 1: a certain amount of raw cow milk is directly sterilized by steam immersion (sterilization temperature is 143-158° C., sterilization duration is 0.05-0.5 s), and quickly cooled to 28° C. after sterilization;
step 2: the Saccharomyces cerevisiae BR14 is activated, specifically including the following steps: inoculating Saccharomyces cerevisiae BR14 into the YPD solid culture medium, and culturing at 28° C. for 48 h to obtain activated Saccharomyces cerevisiae BR14, where the YPD solid medium includes 20 g/L glucose, 20 g/L plant soybean peptone, 10 g/L yeast extract powder and 1.5 wt. % bacterial agar powder, with sterilization at 115° C. for 20 min;
the activated Saccharomyces cerevisiae BR14 is inoculated into 10 mL of liquid culture medium YPD, and cultured at 28° C. for 24 h to obtain the seed liquid of the Saccharomyces cerevisiae BR14; among them, the components of the adopted YPD liquid culture medium are as follows: 20 g/L glucose, 20 g/L plant soybean peptone and 10 g/L yeast, and sterilized at 115° C. for 20 min;
the obtained seed liquid is inoculated into 100 mL YPD liquid culture medium with an inoculation amount of 5% of the volume of YPD liquid culture medium, and cultured at 28° C. and 150 rpm for 12 h; after the culture, the thalli are collected and the OD₆₀₀ value of Saccharomyces cerevisiae BR14 is adjusted to 1 with sterile water;
step 3: inoculation: the Saccharomyces cerevisiae BR14 in step 2 is inoculated into the sterilized cow milk and mixed, where the inoculation amount is 5% of the volume of the sterilized cow milk;
step 4: single-stage fermentation: the fermentation is carried out after evenly stirring, specifically: the rotation speed of the fermentation is controlled at 100 rpm, and the fermentation is carried out for 18 h to obtain the non-acidified fermented milk; the lactose content in the non-acidified fermented milk is 54.2 mg/L, which is 8.61% lower than that of unfermented cow milk, and the pH value is 6.68, with a light milk flavor; and
step 5: the non-acidified fermented milk product after fermentation is homogenized, and then pasteurized at 80° C. for 15 s to obtain the pasteurized non-acidified fermented dairy product.

### Embodiment 7 Effects of Different Yeast on Pasteurized Non-Acidified Fermented Dairy Product

The preparation method of non-acidified fermented dairy products provided by this embodiment is as follows:
step 1: a certain amount of raw cow milk is directly sterilized by steam immersion (sterilization temperature is 143-158° C., and sterilization time is 0.05-0.5 s) and quickly cooled to 28° C. after the sterilization;
step 2: different Saccharomyces cerevisiae strains are activated, and the specific types of Saccharomyces cerevisiae strains are shown in Table 1; the specific steps are as follows: Saccharomyces cerevisiae is inoculated in YPD solid culture medium and cultured at 28° C. for 48 h to obtain activated Saccharomyces cerevisiae; the YPD solid medium includes 20 g/L glucose, 20 g/L plant soybean peptone, 10 g/L yeast extract powder and 1.5 wt. % bacterial agar powder, and is sterilized at 115° C. for 20 min;
the activated Saccharomyces cerevisiae is inoculated into 10 mL of YPD liquid culture medium, and cultured at 28° C. for 24 h to obtain Saccharomyces cerevisiae seed liquid; among them, the components of YPD liquid culture medium are as follows: 20 g/L glucose, 20 g/L plant soybean peptone and 10 g/L yeast, and sterilized at 115° C. for 20 min.
the obtained seed liquid is inoculated into 100 mL YPD liquid culture medium at an inoculation amount is 5% of the volume of YPD liquid culture medium, and cultured at 28° C. and 150 rpm for 12 h; after the culture, the thalli are collected, and the OD₆₀₀ value of Saccharomyces cerevisiae is adjusted to 1 with sterile water;
step 3: inoculation: the Saccharomyces cerevisiae in step 2 is inoculated into the sterilized cow milk and mixed, where the inoculation amount is 5% of the volume of the sterilized cow milk;
step 4: two-stage fermentation: two-stage fermentation is carried out after uniform stirring, specifically: the first-stage fermentation: the rotation speed of the fermentation is controlled at 180 rpm, and fermentation is carried out for 10 h; the second-stage fermentation: the rotation speed of the fermentation is controlled at 100 rpm, and the fermentation is carried out for 8 h to obtain the non-acidified fermented milk; the indexes of the prepared non-acidified fermented milk are shown in Table 1, and the non-acidified fermented milk prepared by Saccharomyces cerevisiae BR14 has the best flavor; and
step 5: the non-acidified fermented milk product after fermentation is homogenized, and then pasteurized at 80° C. for 15 s to obtain the pasteurized non-acidified fermented dairy product.

The lactose, pH and flavor of pasteurized non-acidified fermented dairy products obtained by different strains are investigated, and the lactose, pH and flavor of pasteurized non-acidified fermented dairy product prepared by Embodiment 1 are also investigated, with results as shown in Table 1.

TABLE 1
Effects of different Saccharomyces cerevisiae on non-acidified
fermented dairy product

| | Lactose | | |
|---|---|---|---|
| Bacterial strain | mg/L | pH | Flavor description |
| BR14 (Embodiment | 51.3 | 6.65 | Stronger creamy flavor with |
| 1) | | | slight floral aroma |
| BR49 | 53.1 | 6.72 | Slightly creamy with a slight |
| | | | flavor of decomposed fat |
| BR54 | 55.2 | 6.68 | Good milk flavor |
| BR147 | 52.5 | 6.68 | Slight milk smell |
| BR161 | 54.7 | 6.72 | Slight milk smell |
| BR167 | 53.8 | 6.77 | Good milk flavor with slight |
| | | | lipolysis smell. |
| BR174 | 54.6 | 6.74 | Good milk flavor |

According to the records in Table 1, the fermentation effect of Saccharomyces cerevisiae BR14 is more remarkable, and the lactose content is the lowest among the selected strains, only 51.3 mg/L, and the flavor of fermented milk is the best.

The above-mentioned embodiments only describe the preferred mode of the present disclosure, and do not limit the scope of the present disclosure. Under the premise of not departing from the design spirit of the present disclosure, various modifications and improvements made by ordinary technicians in the field to the technical scheme of the present disclosure shall fall within the protection scope determined by the claims of the present disclosure.

What is claimed is:

1. A preparation method of a non-acidified fermented milk, comprising following steps:
inoculating a fermentation inoculum into cow milk, and performing a two-stage fermentation to obtain the non-acidified fermented milk; wherein
the two-stage fermentation comprises a first-stage fermentation and a second-stage fermentation; a rotation speed of the first-stage fermentation is 180-250 revolutions per minute, and a duration is 10-20 hours; a rotation speed of the second-stage fermentation is 100-160 revolutions per minute, and a duration is 8-16 hours.

2. The preparation method according to claim 1, wherein an active ingredient of the fermentation inoculum is Saccharomyces cerevisiae BR14; and an inoculation amount of the fermentation inoculum is 1%-10% of a volume of the cow milk.

3. The preparation method according to claim 2, wherein an OD₆₀₀ value of the fermentation inoculum is 1.

4. The preparation method according to claim 2, wherein a preparation method of the fermentation inoculum comprises following steps:
activating the Saccharomyces cerevisiae BR14, and then carrying out seed liquid culture and fermentation culture to obtain the fermentation inoculum.

5. The preparation method according to claim 4, wherein a medium used for activating is a YPD solid medium, with a duration of 48 hours and a temperature of 28 degrees Celsius;
a medium used for seed liquid culture is a YPD liquid culture medium, with a duration of 24 hours and a temperature of 28 degrees Celsius; and
the fermentation culture medium adopts the YPD liquid culture medium, with a duration of 12 hours, a temperature of 28 degrees Celsius, a rotation speed of 150 revolutions per minute, and an inoculation amount of 5% of a volume of the YPD liquid culture medium.

6. The preparation method according to claim 1, wherein the cow milk is cow milk obtained after sterilization.

7. The preparation method according to claim 6, wherein a temperature of the sterilization is 143-158 degrees Celsius and a duration is 0.05-0.5 second.

8. A non-acidified fermented milk, wherein the non-acidified fermented milk is obtained by two-stage fermentation of cow milk with Saccharomyces cerevisiae BR14 as a fermentation inoculum.

9. An application of the non-acidified fermented milk according to claim 8 in preparing non-acidified fermented milk products, wherein the products comprise dairy products and freeze-dried powder.

10. The application according to claim 9, wherein when the products are dairy products, pasteurizing the non-acidified fermented milk is also comprised; and
when the products are freeze-dried powder, freeze-drying the non-acidified fermented milk is also comprised.
